(19) United States
(12) Patent Application Publication
(10) Pub. No.: US 2020/0277560 A1
(43) Pub. Date: Sep. 3, 2020

(54) FILTERED CELL CULTURE CAPS AND CELL CULTURE METHODS

(71) Applicant: CORNING INCORPORATED, Corning, NY (US)

(72) Inventor: Jeffery Joseph Scibek, Horseheads, NY (US)

(21) Appl. No.: 16/764,149

(22) PCT Filed: Nov. 29, 2018
(86) PCT No.: PCT/US2018/063022

§ 371 Date: May 14, 2020

Related U.S. Application Data
(60) Provisional application No. 62/592,011, filed on Nov. 29, 2017.

(51) Int. Cl. C12M 1/26; C12M 1/00; C12M 1/02; C12M 1/12; C12N 5/07

(52) U.S. Cl. CPC C12M 33/14; C12M 23/38; C12N 2531/00; C12M 25/06; C12N 5/06; C12M 27/00

(57) ABSTRACT

A bioreactor is provided herein. The bioreactor includes a vessel having a wall at least partially defining an interior compartment for receiving fluid, at least one port, and at least one cap configured to removably engage with the at least one port, the at least one cap comprising a filter material. A cell culture method is also provided herein which includes adding cells and cell growth medium to a vessel of a bioreactor and adding microcarriers to the vessel to form substantially confluent cells on the microcarriers. The cell culture method further includes washing the confluent cells, harvesting the confluent cells to form a solution containing the cells, and removing the solution containing the cells from the vessel by flowing the solution through a filter material in a cap removably engaged with at least one port of the bioreactor.

### CROSS REFERENCE TO RELATED APPLICATIONS

This application claims the benefit of priority of U.S. Provisional Application Ser. No. 62/592,011 filed on Nov. 29, 2017, the contents of which are relied upon and incorporated herein by reference in their entirety as if fully set forth below.

### FIELD

The present disclosure generally relates to cell culture systems and, more particularly, to cell culture systems including a filtered cap for separating cells from microcarriers.

### BACKGROUND

Microcarrier cell culturing is typically carried out in a bioreactor. During culturing, the cells grow on the surface of the microcarriers. Once the cell culturing process is completed, the cultured cells are detached from the microcarriers and the cultured solution containing the cells is then separated from the microcarriers for use or further processing.

A conventional process of detaching cells from the microcarriers includes allowing the microcarriers to settle in the bioreactor. Allowing the microcarriers to settle generally includes discontinuing agitation within the bioreactor. The cell culture media in the bioreactor may then be removed. At least one wash step may then be performed in which a wash solution, containing for example Dulbecco's Phosphate Buffered Saline (DPBS), is added to the bioreactor and then the contents of the bioreactor are agitated for a short period of time. After the short period of agitation, the microcarriers are once again allowed to settle and the wash solution is removed from the bioreactor. A harvest solution which includes a cell detaching agent, such as trypsin, is then added to the bioreactor and agitation is resumed. In combination with the agitation, the harvest solution detaches a substantial number of cells from the microcarriers.

Allowing microcarriers to settle during conventional separation processes of can be time consuming and can, for example, increase the time to complete a conventional separation process by almost 50%. Additionally, settling of cells and microcarriers in the bioreactor can cause aggregation in which the cells and microcarriers become compacted at the bottom of the bioreactor. As a result of becoming compacted, an environment characterized by depleted nutrients and oxygen, high concentration of cellular waste products, and pH extremes may be experienced by the cells. Such an environment can have direct negative effects on cell growth, cell health, and/or cell function.

After detaching the cells from the microcarriers, the microcarriers are conventionally separated from the cultured solution that includes the detached cells. One conventional technique for performing this separation includes passing the solution through a rigid mesh screen in a container. The screen allows the cultured fluid to pass through but prevents the microcarriers from doing so. However, as the microcarriers build up on the screen, they begin to clog the screen and prevent the fluid from passing therethrough. The clogged microcarriers also can trap cells and prevent the cells from passing through the mesh screen. Once the screen is clogged, the process stops until the screen is unclogged. These process steps can be expensive and time consuming and are also believed to contribute to reduced cell yield in microcarrier cell culture. Furthermore, because the mesh screen is a separate system component, the cultured solution must be transferred from the vessel in which the cell culture process is being performed to be passed through the mesh screen. As a result of this transfer, such mesh screens may increase risks of contaminating the cells or the cell culture solution.

Several other techniques for separating microcarriers from the cultured solution that includes the detached cells include, for example, differential gradient centrifugation, acoustic resonance, tangential flow filtration, spin filters and sedimentation using conical or inclined plates. Most of these techniques require expensive capital equipment or are complex to operate.

### SUMMARY

According to embodiments of the present disclosure, a bioreactor is provided. The bioreactor includes a vessel having a wall at least partially defining an interior compartment for receiving fluid, at least one port, and at least one cap configured to removably engage with the at least one port, the at least one cap comprising a filter material.

According to embodiments of the present disclosure, a cell culture method is provided. The cell culture method includes adding cells and cell growth medium to a vessel of a bioreactor and adding microcarriers to the vessel to form substantially confluent cells on the microcarriers. The cell culture method further includes washing the confluent cells, harvesting the confluent cells to form a solution containing the cells, and removing the solution containing the cells from the vessel by flowing the solution through a filter material in a cap removably engaged with at least one port of the bioreactor.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiment(s), and together with the description serve to explain principles and operation of the various embodiments.

### BRIEF DESCRIPTION OF THE DRAWINGS

The disclosure will be understood more clearly from the following description and from the accompanying figures, given purely by way of non-limiting example, in which:

FIG. 1 shows an exemplary bioreactor in accordance with embodiments of the present disclosure;

FIG. 2A is a perspective view of a cap, partially cut-away, having a filter in accordance with embodiments of the present disclosure;

FIG. 2B is a perspective view of a cap having a filter in accordance with embodiments of the present disclosure;

FIG. 3 is a top view of a cap having a filter in accordance with embodiments of the present disclosure;

FIG. 4 is a cut-away perspective view of a bioreactor in accordance with embodiments of the present disclosure;

FIG. 5 is an exploded view of a bioreactor in accordance with embodiments of the present disclosure; and

FIG. 6 illustrates a cell culture method in accordance with embodiments of the present disclosure.

### DETAILED DESCRIPTION

Reference will now be made in detail to the present embodiment(s), an example(s) of which is/are illustrated in the accompanying drawings. Whenever possible, the same reference numerals will be used throughout the drawings to refer to the same or like parts.

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

As used herein, “have,” “having,” “include,” “including,” “comprise,” “comprising” or the like are used in their open ended sense, and generally mean “including, but not limited to.”

All scientific and technical terms used herein have meanings commonly used in the art unless otherwise specified. The definitions provided herein are to facilitate understanding of certain terms used frequently herein and are not meant to limit the scope of the present disclosure.

The present disclosure is described below, at first generally, then in detail on the basis of several exemplary embodiments. The features shown in combination with one another in the individual exemplary embodiments do not all have to be realized. In particular, individual features may also be omitted or combined in some other way with other features shown of the same exemplary embodiment or else of other exemplary embodiments.

Embodiments of the present disclosure relate to bioreactors including sealing caps having a filter in the caps. The caps as described herein allow for separating cells from microcarriers without having to allow time for microcarriers to settle in the bioreactor which in turn reduces the amount of time required to complete the process of separating cells from microcarriers and also reduces the costs associated with performing such a process. Separating cells from microcarriers without having to allow time for microcarriers to settle in the bioreactor also prevents conditions in which the microcarriers become compacted in the bioreactor which in turn reduces or even eliminates the negative effects on cell growth, cell health, and/or cell function associated with microcarriers becoming compacted.

Embodiments of the present disclosure also allow for the process of separating cells from microcarriers to be performed in a single vessel. By facilitating performance of the separation process within a single vessel caps as described herein also reduce contamination risks and cell yield losses associated with removing cells from an initial system or vessel and transferring cells to a subsequent system or vessel. Examples of reduced contamination risks include potential exposure of the cells with contaminants such as, for example, extractables and leachables from the materials of the various systems or vessels and/or particulates which may originate from the materials of the various systems or vessels, or which may originate from the environment during transfer of the cells or cell products from one system or vessel to another system or vessel. Exposure to such contaminants may lead to contamination of costly downstream products which may ultimately need to be discarded as a result of the contamination.

As used herein, the term “fluid” refers to any substance capable of flowing, such as liquids, liquid suspensions, gases, gaseous suspensions, or the like, without limitation. The term “fluid and/or other components” is used throughout the present disclosure to refer to fluid which may include cell culture medium having nutrients for cell growth, cells, byproducts of the cell culture process, and any other biological materials or components that may conventionally be added or formed in a bioprocess system. Vessels described herein may include one or more cells or reagents. The vessels may also include buffers. Additionally, the vessels may include cell culture medium. Cell culture medium may be for example, but is not limited to, sugars, salts, amino acids, serum (e.g., fetal bovine serum), antibiotics, growth factors, differentiation factors, colorant, or other desired factors. Common culture medium that may be provided in the vessels includes Dulbecco's Modified Eagle Medium (DMEM), Ham's F12 Nutrient Mixture, Minimum Essential Media (MEM), RPMI Medium, and the like. Any type of cultured cell may be included in the vessels including, but not limited to, immortalized cells, primary culture cells, cancer cells, stem cells (e.g., embryonic or induced pluripotent), etc. The cells may be mammalian cells, avian cells, piscine cells, etc. The cells may be of any tissue type including, but not limited to, kidney, fibroblast, breast, skin, brain, ovary, lung, bone, nerve, muscle, cardiac, colorectal, pancreas, immune (e.g., B cell), blood, etc. The cells may be in any cultured form in the vessels including disperse (e.g., freshly seeded), confluent, 2-dimensional, 3-dimensional, spheroid, etc. In some embodiments, cells are present without medium (e.g., freeze-dried, in preservative, frozen, etc.).

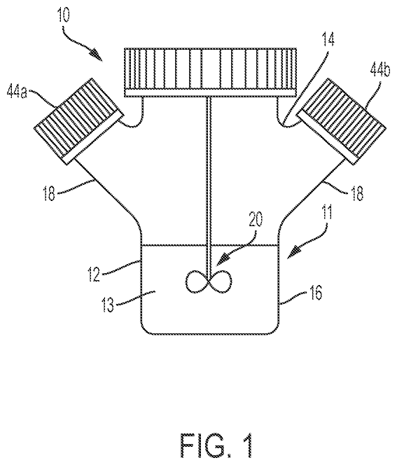

Referring to FIG. 1, a bioreactor in accordance with embodiments of the present disclosure is shown. The bioreactor 10 includes a vessel 11 having a vessel body 12 with a top portion 14 and a bottom portion 16. The vessel 11 also includes necked access ports 18 and an agitator 20 disposed in the interior compartment 13 of the vessel 11. Although two access ports 18 are shown in the figures, it should be appreciated that vessels according to embodiments of the present disclosure may include any number of access ports 18. Each of the necked access ports 18 may be closed by a sealing cap 44a, 44b. The caps 44a, 44b may be internally threaded twist caps configured to cooperate with external threads on the necked access ports 18 of the vessel 11. Additionally, at least one of the caps 44a, 44b may include a filter 210.

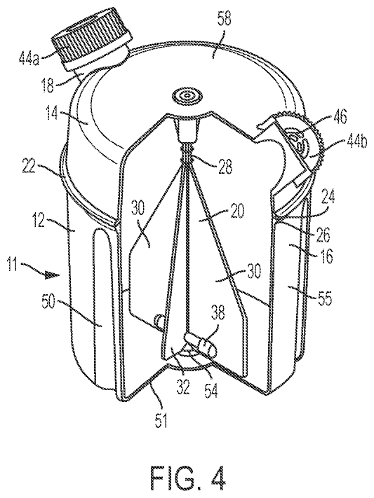

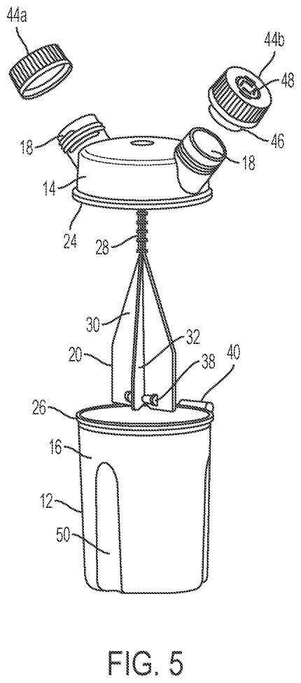

The top portion 14 may include an annular sidewall defining an opening in communication with the interior compartment 13 of the vessel 11. The annular sidewall may have external threads configured to cooperate with internal threads of a twist cap, or the annular sidewall may have an annularly protruding snap cap engagement feature configured to cooperate with a snap cap. Alternatively, the top portion 14 may be integrally formed with the bottom portion 16, or, as shown in FIGS. 4 and 5, may be circumferentially sealed to the bottom portion 16 along a weld line which is the result of joining interconnecting lips circumscribing the periphery of both portions 14, 16.

Bioreactors according to embodiments of the present disclosure may include vessels 10 formed from injection molded polymer, for example polystyrene, polycarbonate, high density polypropylene (HDPE), ultrahigh molecular weight (UHMW) polyethylene, polypropylene, EVA, LDPE and LLDPE or any other polymer as identified by a person of ordinary skill in the art. Optionally, the vessel 11 may be formed from glass, metal or another rigid material.

The vessel 11 may include an agitator 20 in the interior compartment 13 of the vessel 11. The agitator 20 may include a shaft extending from the top portion 14 of the vessel 11, the shaft having at least one impeller along the length of the shaft and being coupled to an overhead motor configured to rotate the at least one impeller within the interior compartment 13 of the vessel 11. Alternatively, the agitator 20 may include a shaft extending from the top of the vessel 11 and having a paddle at the end of the shaft. The shaft is coupled to an overhead motor which is configured to rotate the paddle through a substantially circular path at a nonzero angle relative to a central vertical axis of the vessel 11. An example of such a paddle-based agitator is shown in U.S. Pat. No. 9,168,497 B2. As another alternative, and as shown in FIGS. 4 and 5, the agitator 20 may include a shaft extending from the top of the vessel 11 and having four paddle blades extending from, and contiguous with, the shaft with each of the paddle blades being disposed 90 degrees relative to each other. The four paddle blade agitator further includes a receptacle configured to house a magnetic stir bar which allows for the four paddle blade agitator to be rotated through magnetic induction. An example of such a four paddle blade agitator is shown in U.S. Pat. No. 8,057,092 B2. As yet another alternative, the agitator 20 may include a rotatable impeller disposed in the bottom of the interior compartment 13. The rotatable impeller is at least partially magnetic or ferromagnetic and may be magnetically coupled to an external motive device which includes a rotating drive magnet structure for forming a magnetic coupling with the fluid-agitating element, an electromagnetic structure for rotating and levitating the fluid-agitating element, or a superconducting element for both levitating and rotating the fluid-agitating element. An example of such a rotatable impeller is shown in U.S. Pat. No. 7,481,572 B2.

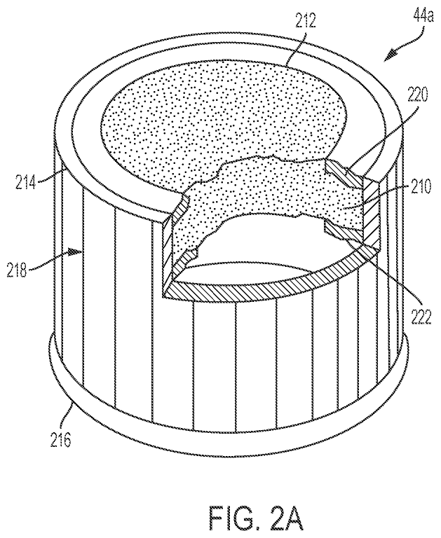

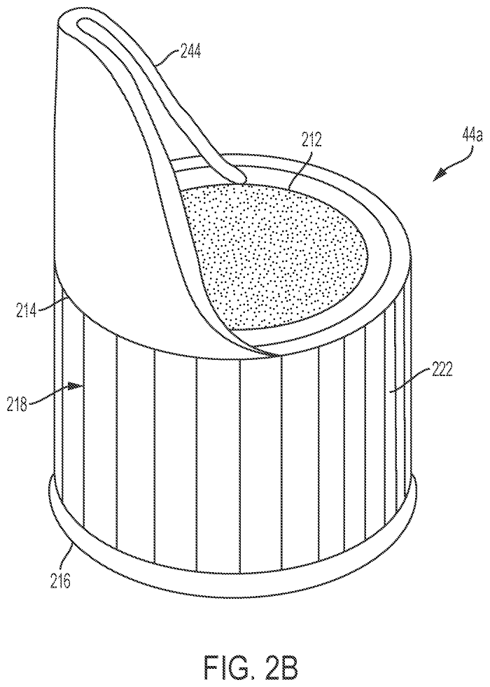

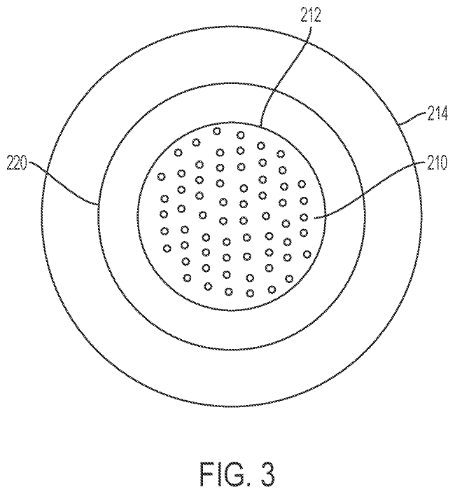

Now referring to FIGS. 2A, 2B and 3, a cap 44a is shown having a filter 210. FIG. 2A shows a partially cut-away view of the cap 44a and FIG. 3 shows a top view of the cap 44a. The filter 210 includes a porous material which allows certain substances to pass out of the vessel 11 while retaining others within the vessel 11. Generally, substances that are small enough to pass through the filter 210 may be those which are regarded as cells or cellular products which may be collected in a container disposed external to the vessel 11 for downstream processing or use. The average pore size of the filter 210 is large enough to allow for the passage of cells, cell culture media and cellular products (e.g., recombinant protein, antibody, virus particles, DNA, RNA, sugars, lipids, biodiesel, inorganic particles, butanol, metabolic byproducts) through the filter 210, but small enough to prevent the passage of microcarriers through the membrane and to retain the microcarriers in the interior compartment 13 of the vessel 11. Generally, the cap 44a may include a filter 210 having an average pore size of between about 1 μm and about 100 μm.

As shown in FIG. 2A, the cap 44a includes a top portion 214 and a bottom portion 216 with a generally cylindrical sidewall 218 extending between the top portion 214 and the bottom portions 216. The generally cylindrical sidewall 218 has an outer surface which may have a variety of surface features formed thereon to provide a secure gripping surface for the cap 44a. The surface feature may be, for example, at least one ridge which protrudes from the outer surface of the sidewall 218. Alternatively, the surface features may take on the form of a series of indentations formed along the outer surface of the sidewall 218.

As shown in the partially cut-away portion of FIG. 2A, the inner surface of the sidewall 218 further includes a lower support plate 222 which secures the filter 210. The lower support plate 222 may extend partially or entirely around the circumference of the inner surface of the sidewall 218, either continuously or in discrete sections. The lower support plate 222 prevents the filter 210 from sliding downward into the interior of the cap 44a. At the top portion 214 of the cap 44a, the filter 210 is secured by an upper support plate 220 which together with the lower support plate 222 form a support structure for the filter 210. FIGS. 2 and 3 further show an opening 212 formed in the top portion 214 of the cap 44a which permits the filter 210 to be exposed to the external environment. In operation, fluid in the interior compartment 13 of a vessel 11 can be brought into contact with an interior side of the filter 210 and pass through the filter 210 and out of the filter through the opening 212 of the cap 44a.

Referring now to FIG. 2B, the cap 44a having a filter 210 as in FIG. 2A is shown further including a pour spout 244. The pour spout 244 may be secured to the top portion 214 of the cap 44a or may be integrally formed with the cap 44a. The pour spout 244 has a funnel-like shape which directs any fluid and/or other components that pass through filter 210 away from cap 44a.

Referring now to FIGS. 4-5, a vessel 11 for cell culture is shown. Like the bioreactor 10 of FIG. 1, the vessel 11 includes a vessel body 12 having a top portion 14 and bottom portion 16, necked access ports 18, and an agitator 20. The top portion 14 and bottom portion 16 are circumferentially sealed along a weld line 22 which is the result of a joining of interconnecting lips 24, 26 circumscribing the periphery of both portions. The vessel 11 has a substantially cylindrical shape with a top surface 58, sidewall 55 and a bottom surface 51 having a centralized raised hump 54.

As shown in FIG. 5, the vessel body 12 includes baffles 50 which extend along the interior wall of the vessel body 12 in a vertical direction which is parallel to the central axis. Each baffle 50 has roughly the cross-sectional shape of a half-cylinder or an isosceles triangle. Each baffle 50 originates from the bottom surface 51 and extends vertically upward terminating in an elliptical shape. The baffles 50 project into the interior compartment of the vessel 11 and, in combination with the agitator 20, create and enable turbulence within the interior compartment of the vessel 11. The vessel 11 shown in FIGS. 4 and 5 include three baffles 50 disposed symmetrically along the interior wall about the central axis, but the number and density of baffles 50 may vary.

The agitator 20 includes a flexible shaft 28 extending along the central axis of the vessel 11. The flexible shaft 28 has a single mounting point on the top portion 14 which permits the shaft 28 to be free to rotate. Extending from and contiguous with the shaft 28 are four paddle blades 30, 32 each disposed about 90 degrees relative to each other. Of the four paddle blades 30, 32 there are two major blades 30 and two minor blades 32. The major blades 30 are disposed about 180 degrees relative to one another and likewise, the two minor blades 32 are disposed about 180 degrees relative to one another. The arrangement of blades around the central shaft creates an alternating effect of minor-major blade orientation. It should be understood that other blade configurations, shapes and arrangements are possible, including those that employ fewer or more than four blades. The agitator 20 may be sized such that the major blades 30 extend nearly the full diameter of the vessel 11. Alternatively, at least one of the major blades 30 may extend about 50% to about 95% or about 75% to about 95%, of the radius of the vessel 11 as measured from the central axis to the sidewall.

The two major blades 30 include a magnet receptacle 38 for receiving a magnetic stir bar 40. A hole in the minor blades 32 and shaft area completes the magnet receptacle 38. A cylindrical plug or magnetic stir bar 40 is mounted in the magnet receptacle 38 along the lower edge of the two major blades 30 and orthogonal to the minor blades 32. Alternatively, the magnet itself may be molded into the agitator 20. To accomplish this, a magnet is inserted into a mold and the agitator is over-molded around the magnet itself

Access ports 18 extend outward from the top portion 14 of the vessel 11. The access ports 18 may be configured to extend from the vessel body 12 at an angle from horizontal to allow instruments to be inserted into the vessel 11 without being restricted by the agitator 20. The dimensions of the access ports 18 and the angles which the access ports 18 extend from the vessel body 12 may be selected to optimize instrument accessibility to various regions within the vessel 11.

Internally threaded sealing caps 44a, 44b may be removably engaged with exterior threads of access ports 18 and may be removed to allow insertion of instruments such as pipettes into the vessel 11. At least one of the caps 44a may include a filter 210 such as the cap 44a shown in FIGS. 2 and 3. Another of the caps 44b may include a hydrophobic membrane insert 46 made from a material that allows gas transport into the vessel interior but prevents liquid from escaping the vessel and other contaminants from entering the vessel. Examples of such membrane material include polytetrafluoethylene and polyvinylidenefluoride (PVDF). Optionally, a cap 44 including a membrane as described herein may further include a vent 48 that allows gaseous communication between the interior of the vessel 11 and the external environment.

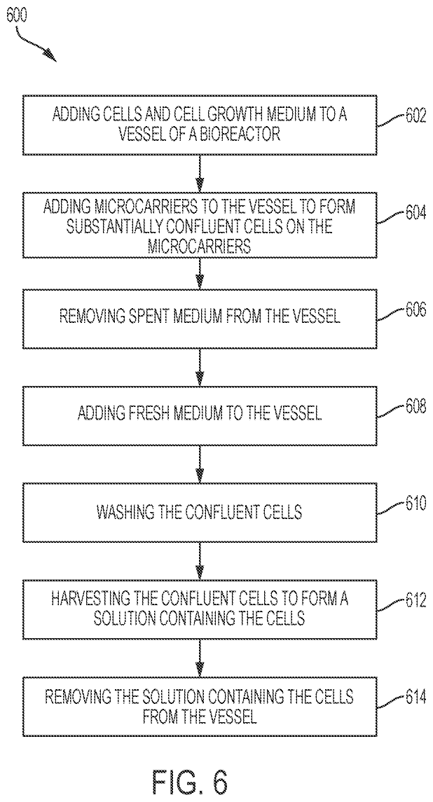

Embodiments of the present disclosure further relate to cell culture methods. FIG. 6 illustrates an exemplary cell culture method according to embodiments of the present disclosure. Such cell culture methods may be performed in bioreactors as described herein and as illustrated in FIGS. 1-5. It should be appreciated that FIG. 6 is merely illustrative of embodiments of the methods described herein, that not all of the steps shown need be performed, and that steps of embodiments of the methods described herein need not be performed in any particular order except where an order is specified.

The cell culture method 600 as described herein may include adding 602 cells and cell growth medium to a vessel 11 of a bioreactor 10. The cell culture method 600 as described herein may further include adding 604 microcarriers to the vessel 11 to form substantially confluent cells on the surfaces of the microcarriers. The microcarriers as described herein may be formed from any material and are conventionally formed from glass materials, plastic materials or hydrogel material. The microcarriers as described herein may have an average diameter of between about 100 micrometers and about 500 micrometers. Commonly, microcarrier have an average diameter of between about 200 micrometers and about 300 micrometers. Any number of microcarriers may be added to the vessel 11 so long as enough microcarriers are added to facilitate substantial confluence in the bioreactor 10. As used herein, the terms “confluent” and “confluence” are used to refer to conditions when cells have formed a coherent monocellular layer on the surface of a cell culture substrate (i.e. the surface of a microcarrier), so that virtually all the available surface is used. For example, “confluent” has been defined as the situation where all cells are in contact all around their periphery with other cells and no available substrate is left uncovered. For purposes of the present disclosure, the term “substantially confluent” is used to refer to conditions when cells are in general contact with the surface of the microcarriers, even though interstices may remain, such that greater than about 70%, or greater than about 80%, or even greater than about 90%, of the available surface is used. The term “available surface” is used to mean sufficient surface area to accommodate a cell. Thus, small interstices between adjacent cells that cannot accommodate an additional cell do not constitute “available surface”.

After cells, medium, and microcarriers are added to the bioreactor 10, growth of the cells within the vessel 11 occurs and continues until the cells occupy the microcarrier surfaces (i.e., until the cells are substantially confluent on the microcarriers), or until the cells exceed the capacity of the medium to support further growth. Exceeding the capacity of the medium is the result of the cells consuming nutrients in the medium and produce waste products which can have direct negative effects on cell growth, cell health, and/or cell function. During the period of the growth of cells at least some portion of the period includes mixing or stirring the fluid and/or other components in the bioreactor 10 with the agitator 20 which causes the microcarriers to become suspended in the interior compartment 13 of the bioreactor 10.

According to embodiments of the present disclosure, the cell culture method 600 as described herein may further include removing 606 spent medium from the vessel 11 by pouring medium from the vessel 11 through the filter 210 of cap 44a. The filter 210 allows for passage of spent medium with cellular products, including cellular waste products, through the filter 210 while retaining microcarriers and confluent cells within the vessel 11. Unlike conventional methods, the filter 210 allows for removal of spent medium without having to allow time for microcarriers to settle toward the bottom surface 51 of the vessel 11. In other words, removal of the spent medium can be accomplished while maintaining the microcarriers in suspension. For the avoidance of doubt, the term “maintaining the microcarriers in suspension” is used to refer to a condition in which the microcarriers are not settled in aggregation in the bioreactor 10. Stirring or mixing with the agitator 20 may be used to maintain the microcarriers in suspension; however, it should be appreciated that stirring or mixing with the agitator 20 may be discontinued and the microcarriers remain in suspension for a period of time after stirring or mixing is discontinued. Therefore, stirring or mixing with the agitator 20 is not required to maintain the microcarriers in suspension. Subsequent to removing 606 spent medium from the vessel 11, the cell culture method 600 as described herein may further include adding 608 fresh medium to the vessel 11. Fresh medium may be added by removing a cap 44a, 44b from the respective necked access port 18 and flowing fresh medium through the opening in the access port 18. The volume of fresh medium added to the vessel 11 may be approximately equal to the volume of spent media removed through the filter 210.

Removing 606 spent medium from the vessel 11 and subsequently adding 608 fresh medium to the vessel 11 may be performed any number of times until the cells are to be separated from the microcarriers and recovered. The cell culture method 600 as described herein may include washing 610 the confluent cells. Prior to washing 610 the confluent cells, the method includes removing 606 spent medium from the vessel 11 without subsequently adding 608 fresh medium to the vessel 11. Washing 610 the confluent cells may include adding a wash solution, containing for example a phosphate buffer solution such as Dulbecco's Phosphate Buffered Saline (DPBS). The washing solution may be added by removing a cap 44a, 44b from the respective necked access port 18 and flowing the washing solution through the opening in the access port 18. With the wash solution in the interior compartment 13 of the vessel 11, the fluid and/or other components in the bioreactor 10 may be mixed or stirred with the agitator 18. Washing 610 the confluent cells may further include removing the washing solution from the vessel 11 and adding a subsequent wash solution. Removing the wash solution from the vessel 11 includes pouring the wash solution from the vessel 11 through the filter 210 of cap 44a. The filter 210 allows for passage of the wash solution through the filter 210 while retaining microcarriers and confluent cells within the vessel 11. Unlike conventional methods, the filter 210 allows for removal of the wash solution without having to allow time for microcarriers to settle toward the bottom surface 51 in the vessel 11. In other words, removal of the wash solution can be accomplished while maintaining the microcarriers in suspension.

Removing the wash solution from the vessel 11 and adding a subsequent wash solution to the vessel 11 may be performed any number of times until the cells are to be separated from the microcarriers. The cell culture method 600 as described herein may further include harvesting 612 the confluent cells to form a solution containing the cells. Prior to harvesting 612 the cells, washing 610 the confluent cells includes removing the wash solution from the vessel 11 without adding a subsequent wash solution to the vessel 11. Harvesting 612 the cells includes adding a harvest solution which may include a detaching agent such as, for example, trypsin to detach the cells from the microcarriers. With the harvest solution in the interior compartment 13 of the vessel 11, the fluid and/or other components in the bioreactor 10 may be mixed or stirred with the agitator 18.

As the cells are removed from the microcarriers a solution containing the cells is formed in the vessel 11 of the bioreactor 10. Optionally, harvesting 612 the cells may further include forming a solution containing the cells within the vessel 11. For example, forming a solution containing the cells may include adding a buffer such as a harvest buffer which maintains an environment (for example, pH conditions) for the cells to remain viable for downstream processing steps, including filtration, capture, and chromatography operations. As another example, the buffer may be a formulation buffer, or a composition which allows for the cells to be used in therapeutic applications after being removed from the vessel 11. Forming a solution containing the cells may also include adding a cryprotectant, or a composition used to protect the cells or cell products from freezing damage, to the vessel 11 such that the cells can be cryopreserved after being removed from the vessel 11.

Following harvesting 612 the cells, the cell culture method 600 as described herein may further include removing 614 the solution containing the cells from the vessel 11. Removing 614 the solution containing the cells includes flowing the solution from the vessel 11 through the filter 210 of cap 44a. The filter 210 allows for passage of the solution containing the cells through the filter 210 while retaining microcarriers within the vessel 11. As previously described in relation to removal of spent medium and removal of the wash solution, removing 614 the solution containing the cells from the vessel 11 can be accomplished while maintaining the microcarriers in suspension.

According to an aspect (1) of the present disclosure a bioreactor is provided. The bioreactor comprises a vessel having a wall at least partially defining an interior compartment for receiving fluid, at least one port, and at least one cap configured to removably engage with the at least one port, the at least one cap comprising a filter material.

According to another aspect (2) of the present disclosure, the bioreactor of aspect (1) is provided further comprising an agitator disposed in the interior compartment of the vessel.

According to another aspect (3) of the present disclosure, the bioreactor of any of aspects (1)-(2) is provided, wherein the at least one port comprises external threads, wherein the at least one cap comprises internal threads, and wherein the internal threads of the at least one cap are configured to cooperate with the external threads of the at least one port.

According to another aspect (4) of the present disclosure, the bioreactor of any of aspects (1)-(3) is provided comprising an injection molded polymer.

According to another aspect (5) of the present disclosure, the bioreactor of any of aspects (1)-(4) is provided, wherein the filter material comprises a porous material having an average pore size of between about 1 μm and about 100 um.

According to another aspect (6) of the present disclosure, the bioreactor of any of aspects (1)-(5) is provided, wherein the at least one cap comprises an opening in the top of the at least one cap which exposes the filter material to the environment external to the vessel.

According to another aspect (7) of the present disclosure, the bioreactor of any of aspects (1)-(6) is provided, wherein the at least one cap comprises an upper support plate disposed above the filter material and a lower support plate disposed below the filter material.

According to another aspect (8) of the present disclosure, the bioreactor of any of aspects (1)-(7) is provided, comprising at least a first port and a second port.

According to another aspect (9) of the present disclosure, the bioreactor of aspect (8) is provided further comprising at least a first cap and a second cap, the first cap being configured to removably engage with the first port and the second cap being configured to removably engage with the second port.

According to another aspect (10) of the present disclosure, the bioreactor of aspect (9) is provided, wherein the first cap comprises a filter material and the second cap does not comprise a filter material.

According to another aspect (11) of the present disclosure, the bioreactor of any of aspects (8)-(9) is provided, wherein the second cap comprises a vent.

According to an aspect (12) of the present disclosure, a cell culture method is provided. The cell culture method comprises adding cells and cell growth medium to a vessel of a bioreactor, adding microcarriers to the vessel to form substantially confluent cells on the microcarriers, washing the confluent cells, harvesting the confluent cells to form a solution containing the cells, and removing the solution containing the cells from the vessel by flowing the solution through a filter material in a cap removably engaged with at least one port of the bioreactor.

According to another aspect (13) of the present disclosure, the cell culture method of aspect (12) is provided, wherein removing the solution containing the cells from the vessel comprises maintaining the microcarriers in suspension.

According to another aspect (14) of the present disclosure, the cell culture method of any of aspects (12)-(13) is provided, further comprising removing spent medium from the vessel by passing medium from the vessel through the filter material of the cap.

According to another aspect (15) of the present disclosure, the cell culture method of aspect (14) is provided, further comprising adding fresh medium to the vessel.

According to another aspect (16) of the present disclosure, the cell culture method of any of aspects (12)-(15) is provided, wherein washing the confluent cells comprises adding a wash solution comprising a phosphate buffer to the vessel.

According to another aspect (17) of the present disclosure, the cell culture method of any of aspects (12)-(16) is provided, wherein washing the confluent cells comprises agitating the contents of the vessel.

According to another aspect (18) of the present disclosure, the cell culture method of any of aspects (12)-(17) is provided, wherein harvesting the confluent cells comprises adding a harvest solution comprising a detaching agent.

According to another aspect (19) of the present disclosure, the cell culture method of aspect (18) is provided, wherein the detaching agent comprises trypsin.

According to another aspect (20) of the present disclosure, the cell culture method of any of aspects (12)-(19) is provided, wherein harvesting the confluent cells comprises agitating the contents of the vessel.

According to another aspect (21) of the present disclosure, the cell culture method of any of aspects (12)-(20) is provided, wherein the microcarriers comprise a material selected from the group consisting of glass, plastic and hydrogel.

According to another aspect (22) of the present disclosure, the cell culture method of any of aspects (12)-(21) is provided, wherein the microcarriers comprise an average diameter of between about 100 micrometers and about 500 micrometers.

According to another aspect (23) of the present disclosure, the cell culture method of any of aspects (12)-(22) is provided, wherein the filter material comprises a porous material having an average pore size of between about 1 μm and about 100 μm.

While the present disclosure includes a limited number of embodiments, those skilled in the art, having benefit of this disclosure, will appreciate that other embodiments can be devised which do not depart from the scope of the present disclosure.

1. A bioreactor comprising:
a vessel having a wall at least partially defining an interior compartment for receiving fluid;
at least one port; and
at least one cap configured to removably engage with the at least one port, the at least one cap comprising a filter material.

2. The bioreactor of claim 1 further comprising an agitator disposed in the interior compartment of the vessel.

3. The bioreactor of claim 1, wherein the at least one port comprises external threads, wherein the at least one cap comprises internal threads, and wherein the internal threads of the at least one cap are configured to cooperate with the external threads of the at least one port.

4. (canceled)

5. The bioreactor of any of the preceding claims claim 1, wherein the filter material comprises a porous material having an average pore size of between about 1 μm and about 100 μm.

6. The bioreactor of claim 1, wherein the at least one cap comprises an opening in the top of the at least one cap which exposes the filter material to the environment external to the vessel.

7. The bioreactor of claim 1, wherein the at least one cap comprises an upper support plate disposed above the filter material and a lower support plate disposed below the filter material.

8. The bioreactor of claims claim 1, comprising at least a first port and a second port.

9. The bioreactor of claim 8, comprising at least a first cap and a second cap, the first cap being configured to removably engage with the first port and the second cap being configured to removably engage with the second port.

10. The bioreactor of claim 9, wherein the first cap comprises a filter material and the second cap does not comprise a filter material.

11. The bioreactor of any claim 8, wherein the second cap comprises a vent.

12. A cell culture method comprising:
adding cells and cell growth medium to a vessel of a bioreactor;
adding microcarriers to the vessel to form substantially confluent cells on the microcarriers;
washing the confluent cells;
harvesting the confluent cells to form a solution containing the cells; and
removing the solution containing the cells from the vessel by flowing the solution through a filter material in a cap removably engaged with at least one port of the bioreactor.

13. The method of claim 12, wherein removing the solution containing the cells from the vessel comprises maintaining the microcarriers in suspension.

14. The method of claim 12, further comprising removing spent medium from the vessel by passing medium from the vessel through the filter material of the cap.

15. The method of claim 14, further comprising adding fresh medium to the vessel.

16. The method of claim 12, wherein washing the confluent cells comprises adding a wash solution comprising a phosphate buffer to the vessel.

17. The method of claim 12, wherein washing the confluent cells comprises agitating the contents of the vessel.

18. The method of claim 12, wherein harvesting the confluent cells comprises adding a harvest solution comprising a detaching agent.

19. The method of claim 18, wherein the detaching agent comprises trypsin.

20. The method of claim 12, wherein harvesting the confluent cells comprises agitating the contents of the vessel.

21. (canceled)

22. The method of claim 12, wherein the microcarriers comprise an average diameter of between about 100 micrometers and about 500 micrometers.

23. The method of claim 12, wherein the filter material comprises a porous material having an average pore size of between about 1 μm and about 100 μm.
